(19) United States
(12) Patent Application Publication
(10) Pub. No.: US 2024/0173024 A1
(43) Pub. Date: May 30, 2024

(54) DRAINAGE CATHETERS WITH LOCKING HUBS CONFIGURED FOR ONE-HANDED PIGTAIL LOCKING OPERATIONS

(71) Applicant: Bard Peripheral Vascular, Inc., Franklin Lakes, NJ (US)

(72) Inventors: Christine T. Kearney, Palatine, IL (US); Nathan P. Retzlaff, Kenosha, WI (US); Michael Plishka, Lake Villa, IL (US); Erin L. Schaus, Elk Grove Village, IL (US)

(21) Appl. No.: 17/994,879

(22) Filed: Nov. 28, 2022

(51) Int. Cl. A61B 17/04

(52) U.S. Cl. CPC A61B 17/04; A61B 2017/0496

(57) ABSTRACT

A drainage catheter includes an elongated tubular body that includes a flexible distal portion and a proximal portion. A locking hub assembly includes a connecting member and an actuation member that is movably connected to the connecting member such that one or both of the actuation member and the connecting member move relative to the other of the actuation member and the connecting member. A tensioning suture extends through a drainage lumen of the tubular body and is connected to the flexible distal portion. Opposite ends of the tensioning suture are connected to the actuation member such that movement of the one or both of the actuation member and the connecting member toward the other of the actuation member and the connecting member from an unlocked configuration to a locked configuration draws the opposite ends proximally relative to the flexible distal portion thereby causing the flexible distal end to move from a loosely curled configuration to a tightly curled configuration.

### TECHNICAL FIELD

The present specification generally relates to drainage catheters and, more specifically, to drainage catheters with pigtail locking hubs configured for one-handed locking operations.

### BACKGROUND

Percutaneous drainage catheters are known that include catheter bodies having distal ends that are configured to curl up into a pigtail to inhibit migration of the catheter and its drainage fenestrations out of an anatomical drainage site target. Many of the catheters have a suture that is utilized to lock the pigtail to further reduce the risk of migration. Typically, these sutures are directly manipulated by a physician pulling on an exposed end of the suture to tighten the suture and then the end is locked into place. However, such locking mechanisms can require two-handed operation (one hand to hold the catheter and a second hand to tighten the suture). Locking procedures themselves may further require two hands to lock the suture at a desired location.

What is needed are drainage catheters with locking hubs that are configured for one-handed pigtail locking operations.

### SUMMARY

According to a first embodiment, a drainage catheter includes an elongated tubular body that includes a flexible distal portion and a proximal portion. A locking hub assembly includes a connecting member and an actuation member that is movably connected to the connecting member such that one or both of the actuation member and the connecting member move relative to the other of the actuation member and the connecting member. A tensioning suture extends through a drainage lumen of the tubular body and is connected to the flexible distal portion. Opposite ends of the tensioning suture are connected to the actuation member such that movement of the one or both of the actuation member and the connecting member toward the other of the actuation member and the connecting member from an unlocked configuration to a locked configuration draws the opposite ends proximally relative to the flexible distal portion thereby causing the flexible distal end to move from a loosely curled configuration to a tightly curled configuration.

In another embodiment, a drainage catheter includes An elongated tubular body that includes a flexible distal portion and a proximal portion. A locking hub assembly includes a connecting member and an actuation member movably connected to the connecting member such that one or both of the actuation member and the connecting member move relative to the other of the actuation member and the connecting member. A tensioning suture extends through a drainage lumen of the tubular body and is connected to the flexible distal portion. The connecting member includes a shell having a suture hole extending therethrough that is in communication with a drainage channel of the shell. The tensioning member is threaded through the suture hole providing an anchoring location for the tensioning suture. An end of the tensioning suture is connected to the actuation member such that movement of the one or both of the actuation member and the connecting member toward the other of the actuation member and the connecting member from an unlocked configuration to a locked configuration draws the end proximally relative to the flexible distal portion thereby causing the flexible distal end to move from a loosely curled configuration to a tightly curled configuration.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a drainage catheter that includes a locking hub assembly in an unlocked configuration, according to one or more embodiments shown and described herein;

FIG. 2 schematically depicts the drainage catheter of FIG. 1 with the locking hub assembly in a locked configuration, according to one or more embodiments shown and described herein;

FIG. 3 schematically depicts a distal portion of a tubular body of the drainage catheter of FIG. 1 in a pigtail configuration, according to one or more embodiments shown and described herein;

FIG. 4 schematically depicts the locking hub assembly of FIG. 1 in isolation in the unlocked configuration, according to one or more embodiments shown and described herein;

FIG. 5 schematically depicts the locking hub assembly of FIG. 4 in the locked configuration, according to one or more embodiments shown and described herein;

FIG. 6 schematically depicts a section view of the locking hub assembly of FIG. 5, according to one or more embodiments shown and described herein;

FIG. 7 schematically depicts another locking hub assembly, according to one or more embodiments shown and described herein;

FIG. 8 schematically depicts a section view of the locking hub assembly of FIG. 7 in a locked configuration, according to one or more embodiments shown and described herein;

FIG. 9 schematically depicts the locking hub of FIG. 8 in a squeezed, deflected configuration, according to one or more embodiments shown and described herein;

FIG. 10 schematically depicts another locking hub assembly, according to one or more embodiments shown and described herein;

FIG. 11 schematically depicts another locking hub assembly in an unlocked configuration, according to one or more embodiments shown and described herein;

FIG. 12 schematically depicts the locking hub assembly of FIG. 11 in a locked configuration, according to one or more embodiments shown and described herein;

FIG. 13 schematically depicts a section view of the locking hub assembly of FIG. 11, according to one or more embodiments shown and described herein;

FIG. 14 schematically depicts a section view of the locking hub assembly of FIG. 12, according to one or more embodiments shown and described herein;

FIG. 15 schematically depicts a partial view of a proximal end of the locking hub assembly of FIG. 8, according to one or more embodiments shown and described herein;

FIG. 16 schematically depicts another locking hub assembly in an unlocked configuration, according to one or more embodiments shown and described herein;

FIG. 17 schematically depicts the locking hub assembly of FIG. 16 in a locked configuration, according to one or more embodiments shown and described herein;

FIG. 18 schematically depicts the locking hub assembly of FIG. 16 with a tensioning suture connected to the actuation member, according to one or more embodiments shown and described herein;

FIG. 19 schematically depicts the locking hub assembly of FIG. 18 with the actuation member rotated, according to one or more embodiments shown and described herein; and

FIG. 20 schematically depicts a section view of the locking hub assembly of FIG. 17, according to one or more embodiments shown and described herein.

### DETAILED DESCRIPTION

Embodiments described herein are generally directed to drainage catheters that may that include locking hubs that can be operated using one hand instead of generally requiring two hands for pigtail locking operations. The drainage catheters include an elongated tubular body that includes a flexible distal portion and a proximal portion. A locking hub assembly includes an actuation member that is connected to the proximal portion of the tubular body and a connecting member that is movably connected to the actuation member such that one or both of the actuation member and the connecting member move relative to the other of the actuation member and the connecting member. A tensioning suture extends through a drainage lumen of the tubular body and is connected to the flexible distal portion. Opposite ends of the tensioning suture are connected to the actuation member such that movement of the one or both of the actuation member and the connecting member relative to the other of the actuation member and the connecting member draws the opposite ends proximally relative to the flexible distal portion thereby causing the flexible distal end to move from a loosely curled configuration to a tightly curled configuration.

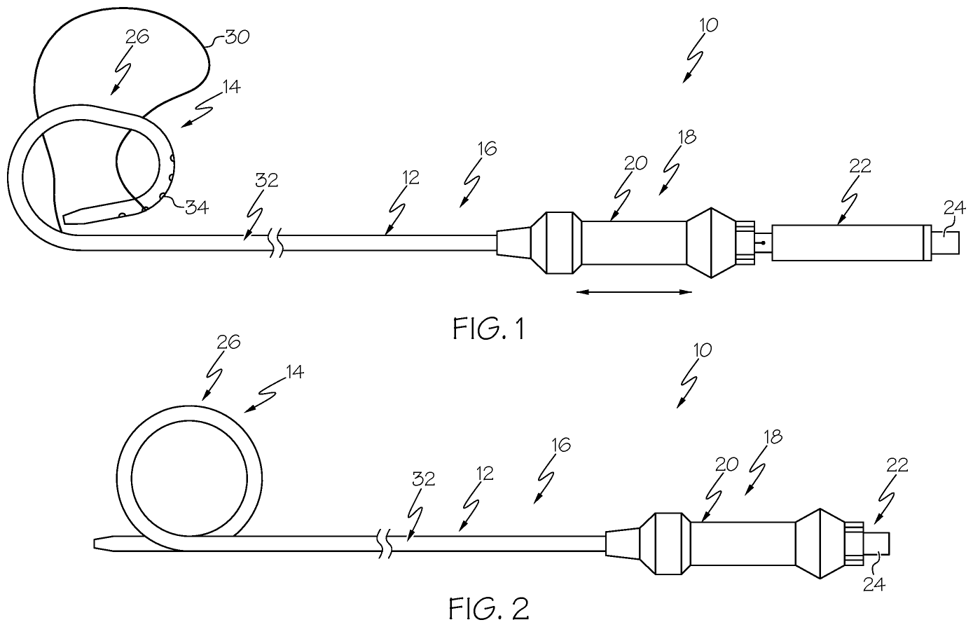

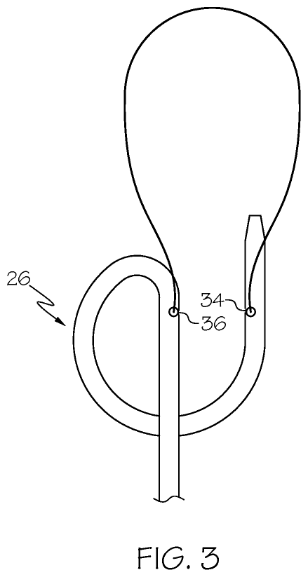

Referring to FIGS. 1 and 2, a drainage catheter 10 includes an elongated tubular body 12 that includes a distal portion 14 and a proximal portion 16. In some embodiments, both the distal portion 14 and the proximal portion 16 are flexible so that the tubular body 12 may bend and flex easily under manual force and under the force of gravity. The proximal portion 16 is fluidly connected to a locking hub 18. The locking hub 18 includes an actuation member 20 that is fluidly connected to the proximal portion 16 of the tubular body 12 and a connecting member 22 that is movably connected to the actuation member 20. The actuation member 20 and the connecting member 22 are movably connected together such that one or both of the actuation member 20 and the connecting member 22 moves relative to the other of the actuation member 20 and the connecting member 22. The connecting member 22 includes a connector 24, such as a Luer connector that can be connected to another device, such as a straightening cannula that can be threaded through the locking hub 18 and the tubular body 12 or a drainage container for collecting bodily fluid during a drainage operation. The straightening cannula may include a corresponding connector and a conduit that can be used for straightening a pigtail 26, which is a curved configuration of the distal portion 14, as described in more detail in U.S. Pat. No. 5,472,435 titled “Drainage Catheter,” filed May 21, 1993, the details of which are incorporated by reference in their entirety. For example, the distal portion 14 may be formed having a curve or loosely coiled configuration when not straightened as shown by FIG. 1. In these embodiments, when no force is applied to the distal portion 14, the distal portion 14 is curved into the pigtail shape.

The drainage catheter 10 further includes a tensioning suture 30 that extends through a drainage lumen 32 of the tubular body 12. The tensioning suture 30 may be connected to the actuation member 20, extend distally through the drainage lumen 32 and exit a side port 34 at the distal portion 14 of the tubular body 12. The tensioning suture 30 may then re-enter the drainage lumen 32 through another side port 36 (FIG. 3) and travels proximally back to the actuation member 20 as will be described below.

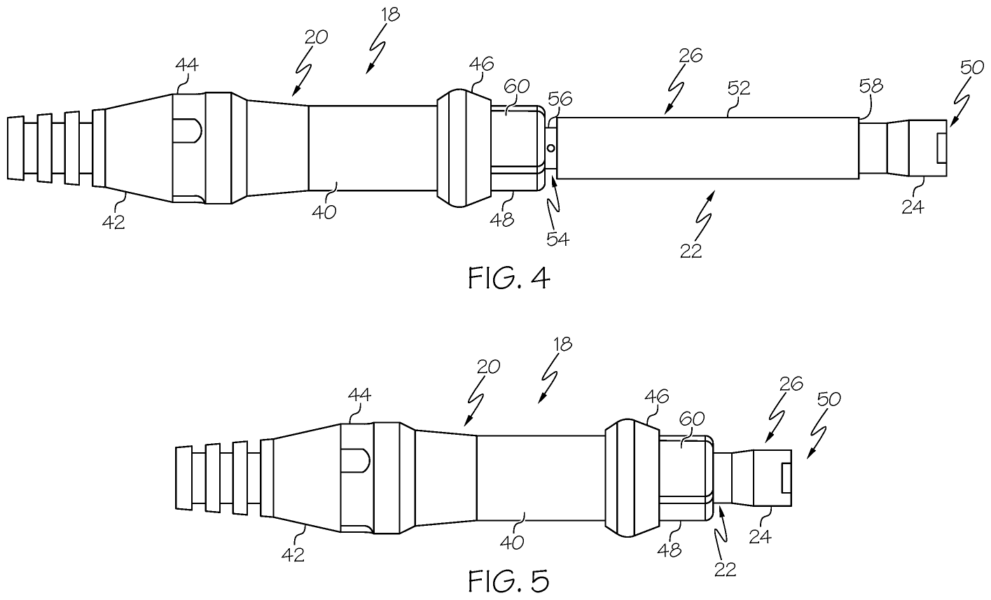

FIGS. 4 and 5 illustrate the locking hub 18 in isolation showing an unlocked configuration (FIGS. 1 and 4) and a locked configuration (FIGS. 2 and 5). The locking hub 18 includes the actuation member 20 and the connecting member 22. The actuation member 20 and connecting member 22 are slidably connected together such that the actuation member 20 can slide over the connecting member 22 in a telescoping fashion. The actuation member 20 connects directly to the tubular body 12 and includes an outer sliding shell 40, a strain relief member 42 that is connected to a distal end 44 of the outer sliding shell 40 and a pulling ring 46 that is disposed around a proximal end 48 of the outer sliding shell 40. The strain relief member 42 connects to the tubular body 12 and provides the tubular body 12 with support against sharp bends at the connection between the tubular body 12 and the actuation member 20. The pulling ring 46 may provide an anchor point for two fingers to pull against in order to facilitate one-handed locking with a thumb placed on a proximal end 50 of the connecting member 22.

Referring particularly to FIG. 4, the connecting member 22 connects indirectly to the tubular body 12 through its connection to the actuation member 20 and includes an inner sliding shell 52 with a necked-down region 54 and the connector 24 located at the proximal end 50. A suture hole 56 is provided in the necked-down region through which the tensioning suture 30 extends in order to connect to the outer sliding shell 40. The connecting member 22 may also include a locking step 58 located at the proximal end 50 against which a corresponding mating connection 60 of the outer sliding shell 40 can mate. For example, the connection 60 may be a snap fit connection that snaps against the locking step 58 forming an interference fit and providing one or both of an audible and tactile feedback to the user when the actuation member 20 is placed in the locked configuration.

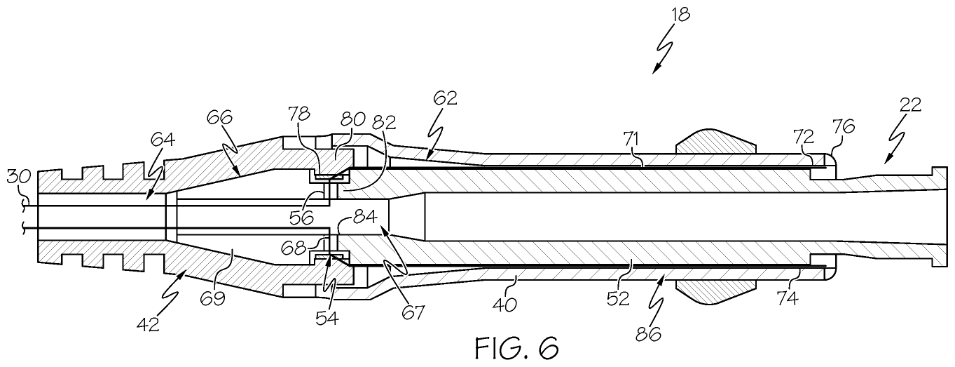

Referring now to FIG. 6 showing a section view of the locking hub 18 in the locked configuration, the connecting member 22 is slidingly received within an actuation channel 62 of the actuation member 20. The actuation channel 62 is formed by the outer sliding shell 40 and also extends through a tube receiving bore 64 of the strain relief member 42. A flared portion 66 extends outward from the tube receiving bore 64 that is sized to receive a tapered distal end 69 of the connecting member 22 in the locked configuration.

The tensioning suture 30 extends from the distal portion 14 of the tubular body 12 through the drainage lumen 32 and the tube receiving bore 64 and into an inner drainage channel 67 of the connecting member 22. The tensioning suture 30 is then threaded through the suture holes 56 and 68 where the tensioning suture 30 may then travel proximally along an exterior surface 71 of the connecting member 22 to opposite terminal ends 72 and 74. An elastomer sealing member 78 (e.g., an elastomer ring) may be provided around the necked-down region 54 to aid in sealing the suture holes 56 and 68 in the locked configuration. In the illustrated example, the strain relief member 42 includes a sealing lip 80 that has a reduced inner diameter in order to be received within the necked-down region 54 and engage against the sealing ring 78 in the locked configuration in order to seal against the suture holes 56 and 68 and inhibit leaking of bodily fluids through the suture holes 56 and 68 during a drainage procedure.

The tensioning suture 30 may then be connected to the outer sliding shell 40 using any suitable connection. As one example, the outer sliding shell 40 may be formed of a thermoplastic material (e.g., acrylonitrile butadiene styrene) having a lower melting point than the material forming the tensioning suture 30 (e.g., nylon) such that the outer sliding shell 40 can be molded onto the tensioning suture 30. In this regard, the opposite ends 72 and 74 of the tensioning suture 30 may be embedded into the outer sliding shell 40. While the opposite ends 70 and 72 are shown near to a proximal end 76 of the outer sliding shell 40, they may be located at any suitable location along the length of the outer sliding shell 40, which can depend on, for example, the size of the distal portion 14 of the tubular body 12 and shape of the pigtail.

Because the tensioning suture 30 is threaded through the suture holes 56 and 68, the inner sliding shell 52 forms two anchoring locations 82 and 84 about which the tensioning suture 30 extends that sets two fixed locations for the tensioning suture 30 relative to the inner sliding shell 52. As used herein, an “anchoring location” is a location about which the tensioning suture 30 is trained and changes its direction of extension from a straight line to a curved line. It should be noted that in embodiments where only one end 72, 74 of the tensioning suture 30 is connected to the outer sliding shell 40, only one anchoring location 82, 84 may be provided by the outer sliding shell 40. When the outer sliding shell 40 is moved toward and proximally over the inner sliding shell 52, the opposite ends 72 and 74 can move past the anchoring locations 82 and 84, thereby increasing a length L from the anchoring locations 82 and 84 to the opposite ends 72 and 74. Thus, 2 L is the length of the tensioning suture 30 that is taken up from the distal portion 14 thereby tightening the pigtail as represented by FIG. 2.

Various channels, cutouts and snap fits can be used to lock the actuation member 20 and the connecting member 22 in the locked configuration. The actuation member 20 may also be rotated in various directions in addition to pushing and pulling. Rotation can also make it easier or more difficult to lock and unlock the locking hub 18. In some embodiments, there is a level of interference fit between the outer sliding shell 40 and the inner sliding shell 52. Further, the outer sliding shell 40 does not need to be entirely solid, which can improve patient comfort. For example, a distal portion of the outer sliding shell 40 represented by element 86 may be flexible, e.g., from the pulling ring 46 to the strain relief member 42. When the locking hub 18 is in the unlocked configuration (FIGS. 1 and 4), this flexible area may crumple (i.e., reduce in size) and then be pulled taut when the locking hub 18 is placed in the locked configuration.

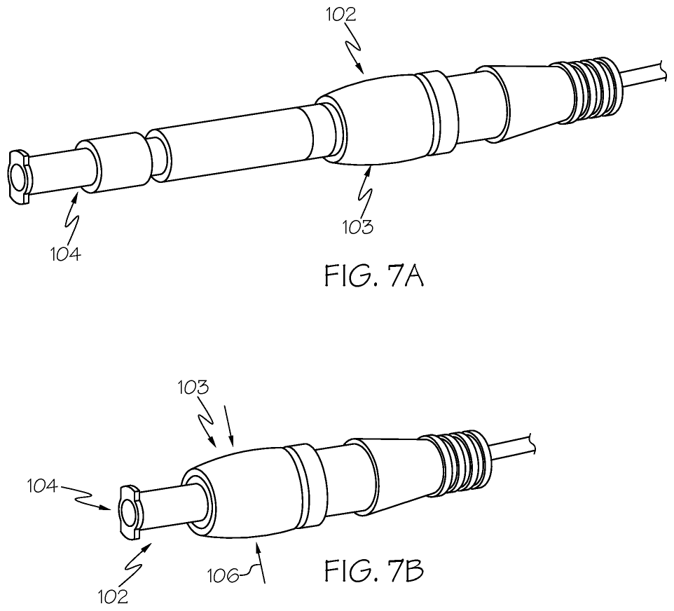

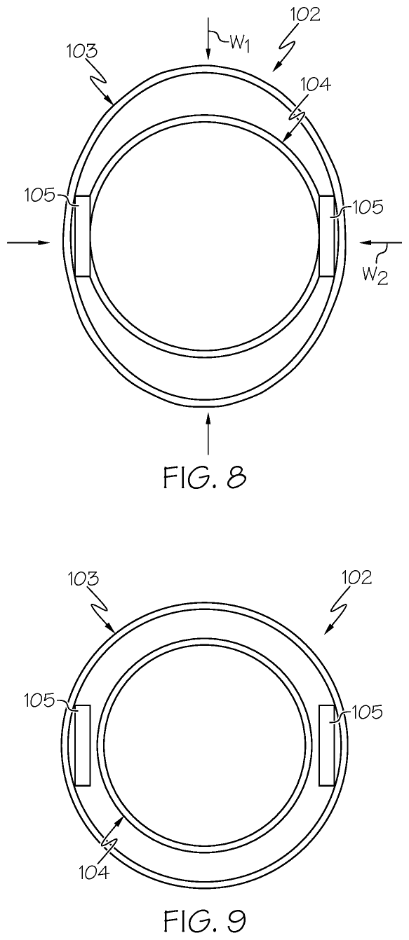

Referring to FIGS. 7-9, a variation of the drainage catheter 10 includes an asymmetric locking hub 102 whose actuation member 103 can be squeezed or otherwise deflected in order to disengage the actuation member 103 and connecting member 104. In particular, the actuation member 103 may be formed oblong in that a width W₁ in one direction may be longer than a width W₂ in a direction perpendicular to W₁. When the locking hub 102 is placed in the locked configuration as shown by FIG. 8, engaging elements 105 of the actuation member 103 engage the connecting member 104. When it is desired to unlock the locking hub 102, the actuation member 103 can be squeezed as represented by the arrows 106 of FIG. 7, which disengage the engaging elements 105 from the connecting member 104 ant the actuation member 103 and connecting member 104 can be slid apart to the unlocked configuration. The actuation member 103 may be formed of a resiliently flexible material, such as by overmolding to be manually deformable.

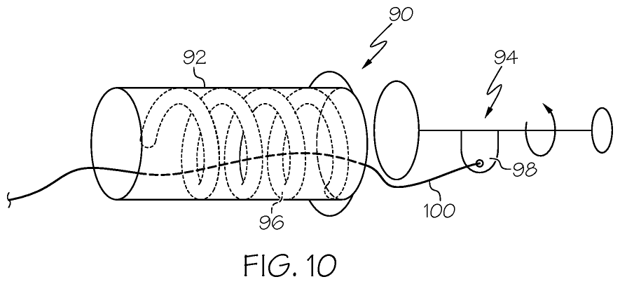

Referring to FIG. 10, another embodiment of a locking hub 90 uses an actuation member 92 and a connecting member 94 that spins as the actuation member 92 is drawn over the connecting member 94. In this example, the actuation member 92 may include a track 96, such as a channel or trench that is spiral in shape and along which a follower element 98 (e.g., a paddle) travels as the actuation member 92 is pulled linearly. A tensioning suture 100 may be attached to the follower element 98 that is taken up by the rotational travel of the follower element 98. The locking hub 90 can include any of the features described above with regard to locking hub 18. Taking up the tensioning suture 100 through rotational travel of the follower element 98 may optionally allow increased uptake of the tensioning suture for a given linear movement of the actuation member 92 relative to the connecting member 94. This may allow a locking hub 90 to be shorter overall while providing a given amount of uptake of the tensioning suture.

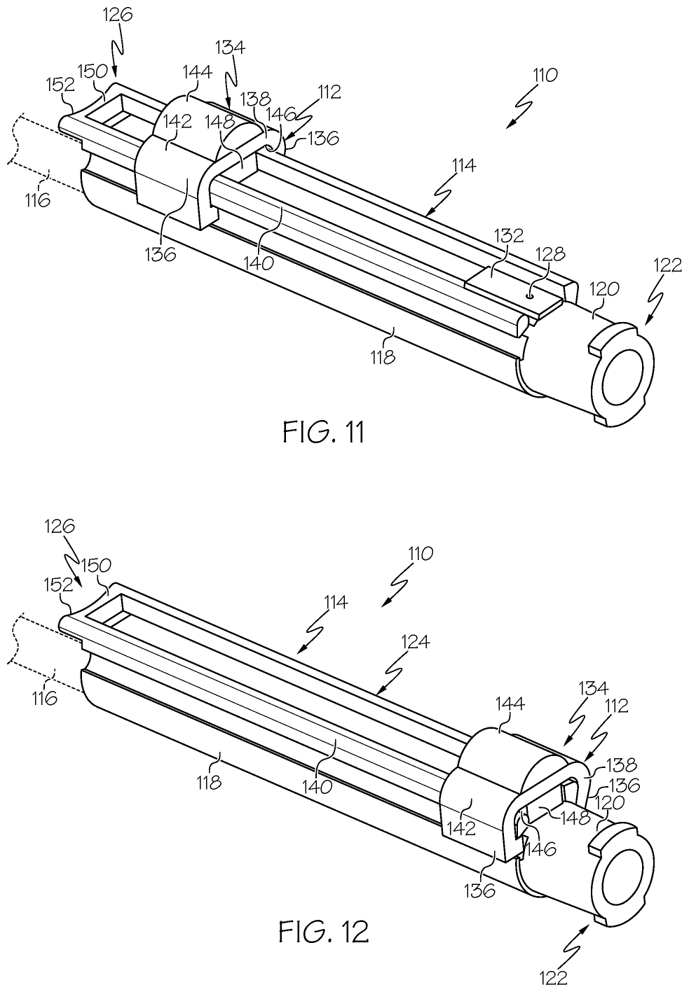

Referring to FIGS. 11 and 12, another embodiment of a locking hub 110 includes an actuation member 112 and a connecting member 114. FIG. 11 illustrates the locking hub 110 in an unlocked configuration (without a tensioning suture) with the actuation member 112 moved to a distal position relative to the connecting member 114 and FIG. 12 illustrates the locking hub 110 in a locked configuration with the actuation member 112 moved to a proximal position relative to the connecting member 114. In this embodiment, the connecting member 114 connects directly to a tubular body 116 that is similar to or the same as the tubular body 12 described above and the actuation member 112 is indirectly connected to the tubular body 116 through its connection with the connecting member 114.

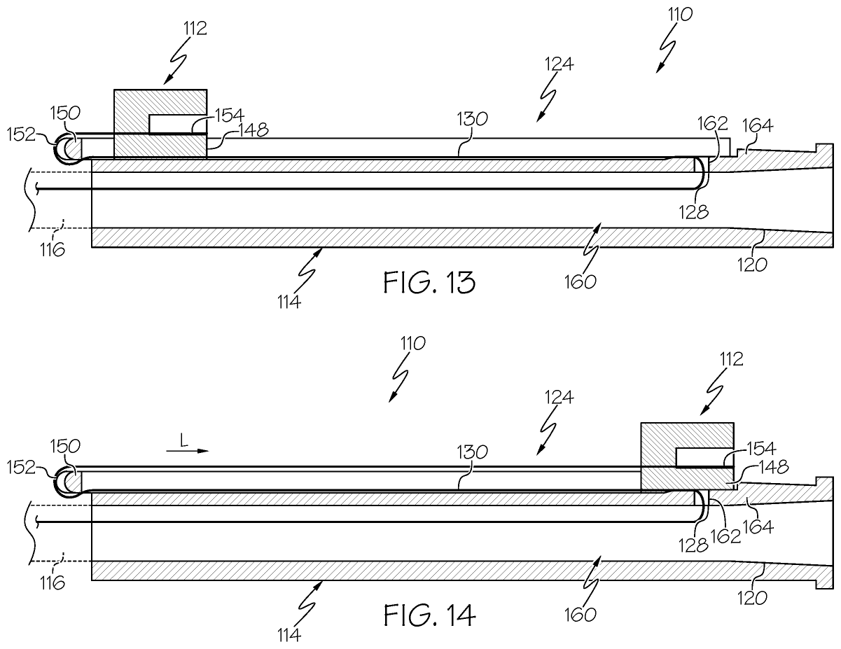

The connecting member 114 includes a shell 118 with a connector 120 located at proximal end 122. The shell 118 includes a sliding track 124 that extends longitudinally from the proximal end 122 to a distal end 126. Located within the sliding track 124 is a suture hole 128 through which a tensioning suture 130 (FIGS. 13 and 14) extends in order to connect to the actuation member 112. In some embodiments, a sealing member 132 is provided around the suture hole 128 to help seal the suture hole 128 with the locking hub 110 in the locked configuration (FIG. 12).

The actuation member 112 includes a sliding ring 134 that is slidably connected to the sliding track 124. In particular, the sliding ring 134 may include hooking arms 136 on opposite sides of a cover 138, where the hooking arms 136 hook underneath a pair of sliding rails 140 that extend the length of the sliding track 124 and the cover extends over both sliding rails 140. In some embodiments, an outer face 142 of the cover 138 may include a pulling member 144 that can aid in engaging and moving the actuation member 112 relative to the connecting member 114. An inner face 146 of the cover 138 may include an elastomer engaging member 148 that is used to engage against the sealing member 132 with the locking hub 110 in the locked configuration.

An anchoring bar 150 extends laterally between the sliding rails 140 and is located at the distal end of the sliding track 124. The anchoring bar 150 provides an anchoring location 152 about which the tensioning suture 130 (FIGS. 13 and 14) extends that sets a fixed location for the tensioning suture 130 relative to the shell 118. In the embodiment of FIG. 13, only one of the opposite ends 154 of the tensioning suture 130 is connected to the actuation member 112, such as by embedding the tensioning suture 130 in the actuation member 112 as described above. Where both opposite ends 154 are connected to the actuation member 112, the anchoring bar 150 provides two anchoring locations 152.

Referring to FIGS. 13 and 14, a section view of the locking hub 110 is illustrated in the unlocked configuration (FIG. 13) and the locked configuration (FIG. 14) with the tensioning suture 130 present. The tensioning suture 130 is routed through the tubular body 116 from the pigtail (FIGS. 1 and 2) to a drainage channel 160 that extends through the connecting member 114 to the connector 120. The tensioning suture 130 is routed through the suture hole 128 which provides a first anchoring location 162 for the tensioning suture 130. The tensioning suture 130 is then routed distally along the sliding track 124 to the anchoring bar 150 and then around the anchoring bar 150 providing the second anchoring location 152. The end 154 of the tensioning suture 130 is connected to the actuation member 112.

Referring to FIG. 14, as the actuation member 112 is moved proximally away from the tubular body 116, a length L of the tensioning suture 130 from the second anchoring location 152 increases. Thus, in this embodiment, L is the length that the tensioning suture 130 that is taken up from the distal portion 14 thereby tightening the pigtail as represented by FIG. 2. Where both opposite ends 154 of the tensioning suture 130 are connected to the actuation member 112, 2 L is the length that the tensioning suture 130 is taken up from the distal portion 14. In the locked configuration, the engaging member 148 can engage the sealing member 132 (FIG. 11) thereby sealing the suture hole 128. The engagement with the sealing member 132 can also provide one or both of a tactile and audible feedback when the actuation member 112 is placed in the locked configuration. Further, a stop 164 may be provided to limit a proximal travel distance of the actuation member 112 toward the connector 120. The anchoring bar 150 may likewise provide distal stop that limits a distal travel distance of the actuation member 112 toward the tubular body 116.

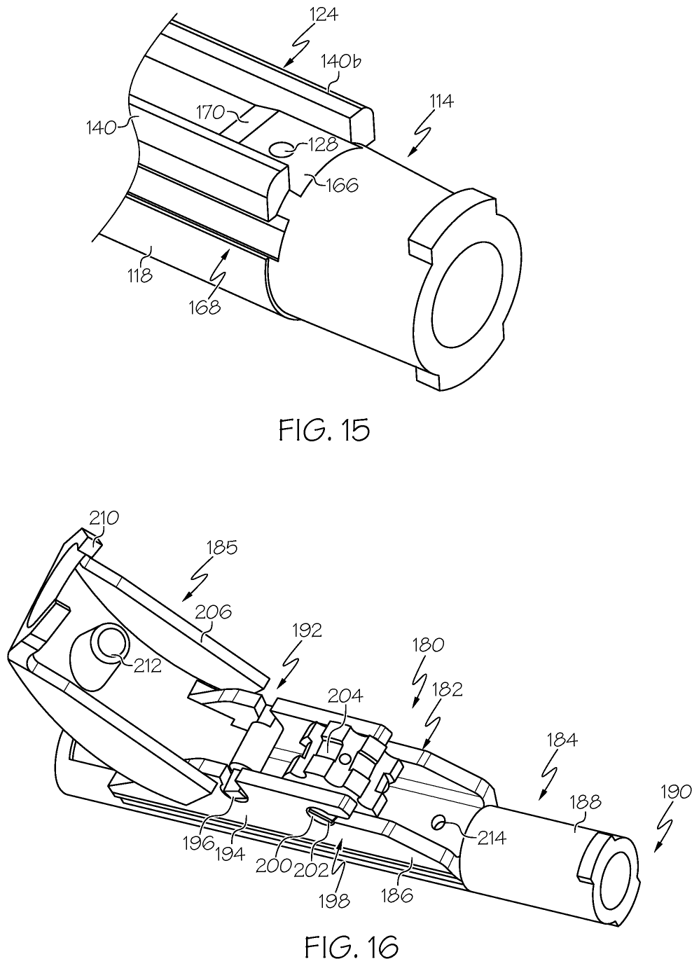

FIG. 15 illustrates a more detailed view of the locking geometries of the connecting member 114 with the sealing member 132 removed. Longitudinally-extending recesses 168 may be provided under and alongside the sliding rails 140a and 140b that can each receive a respective hooking arm 136 of the actuation member 112 and inhibit radial movement of the actuation arm off the sliding track 124. The suture hole 128 may be located at a raised portion 166 that is located between the sliding rails 140a and 140b of the sliding track 124. A locking ramp 170 may be provided such that, when the actuation member 112 is slid proximally, the locking ramp 170 can create additional pressure to lock the position of the actuation member 112 against the connecting member 114 and also provide additional sealing pressure.

While the suture travel is depicted as being partially external to the shell 118 along the sliding track 124, additional channels can be provided such that the tensioning suture 130 is not exposed. Other locking geometries may be used, such as cuts, detents and bosses that engage with the actuation member 112 to lock it into place when in the locked configuration.

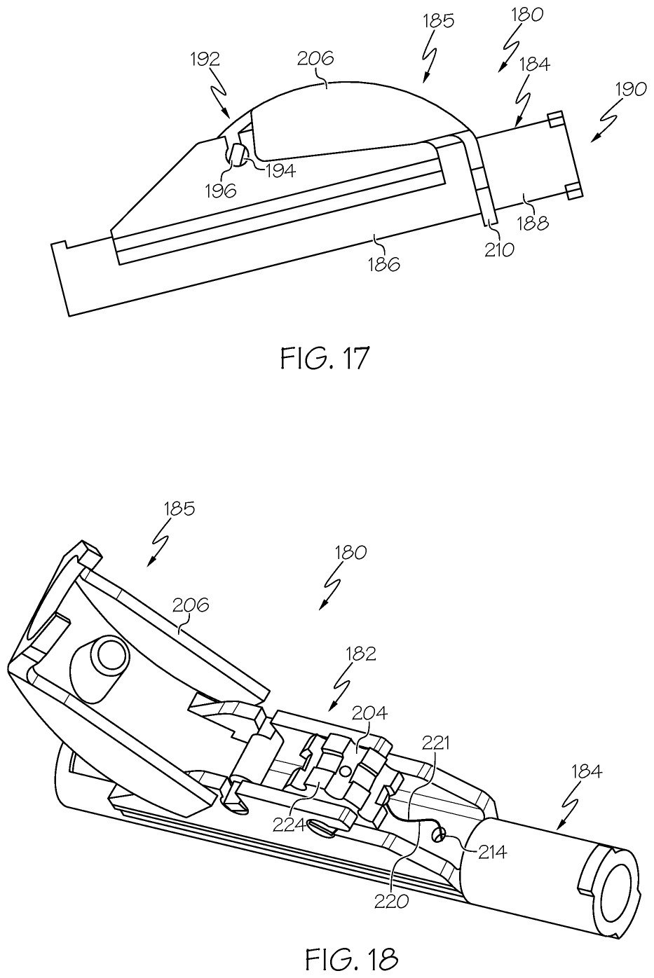

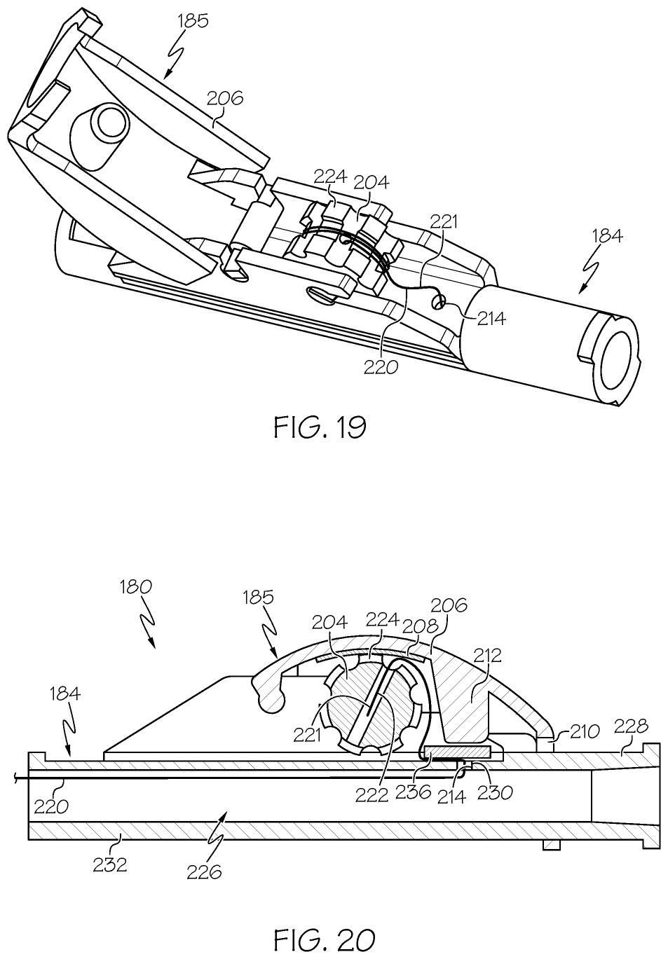

Referring to FIGS. 16-20, another embodiment of a locking hub 180 includes an actuation member 182 and a connecting member 184. FIG. 16 illustrates the locking hub 180 in an unlocked configuration (without a tensioning suture) with a locking cover 185 in an open position and FIG. 17 illustrates the locking hub 180 in a locked configuration with the locking cover in a closed position. In this embodiment, the connecting member 184 connects directly to a tubular body that is similar to or the same as the tubular body 12 described above and the actuation member 182 is indirectly connected to the tubular body through its connection with the connecting member 184.

The connecting member 184 includes a shell 186 with a connector 188 located at proximal end 190. The shell 186 includes a first pivot connection 192 that includes recesses 194 for receiving a pivot rod 196 of the locking cover 185 and a second pivot connection 198 that includes recesses 200 for receiving a pivot rod 202 of the actuation member 182 that comprises a rotating wheel 204.

The locking cover 185 may include a cover body 206 that is shaped to extend over the actuation member 182 and also to engage the rotating wheel 204 and lock it from spinning. In some embodiments, referring to FIG. 20, the cover body 206 may include a locking member 208, such as an elastomer pad, that can frictionally engage the rotating wheel 204 and inhibit spinning. The locking cover 185 may further include a shell engaging member 210, such as a clamp that can clamp about the shell 186 (FIG. 16) and hold the locking cover 185 in the locked configuration. The locking cover 185 may also include a sealing boss 212 that can engage the shell 186 at a suture hole 214 for sealing the suture hole 214. In some embodiments, the boss 212 may include a sealing member, such as an elastomer pad, to seal against the shell 186 and around the suture hole 214. The sealing member may optionally prevent or reduce leakage through the suture hole 214.

Referring particularly to FIGS. 18-20, the rotating wheel 204 of the actuation member 182 is connected to an end 221 of a tensioning suture 220. As an example, the section view of FIG. 20 illustrates the rotating wheel 204 with an opening 222 that can receive the end 221 of the tensioning suture 220. The rotating wheel 204 can have friction enhancing projections 224 that can improve manual engagement with the rotating wheel 204.

Referring to FIG. 20, the tensioning suture 220 is routed through the tubular body from the pigtail (FIGS. 1 and 2) to a drainage channel 226 that extends through the connecting member 184 to a connector 228. The tensioning suture 220 is routed through the suture hole 214 which provides an anchoring location 230 for the tensioning suture 220. The tensioning suture 220 is then routed distally along shell 232 to the rotating wheel 204. The end 221 of the tensioning suture 220 is connected to the rotating wheel 204 using the opening 222. In another embodiment, the end 221 of the tensioning suture 220 may be embedded in the rotating wheel 204. As can be appreciated, opening the locking cover 185 and rotating the rotating wheel 204 can take up a desired length of the tensioning suture 220. Once a desired amount of length is wrapped around the rotating wheel 204, the locking cover 185 can be closed such that the locking member 208 engages the rotating wheel 204 preventing it from spinning and the sealing boss 212 engages a sealing member 236 extending around the suture hole 214. This may optionally allow a user to roll the rotating wheel 204 using a finger or thumb, to obtain the desired level of tensioning of the tensioning suture 220, before locking in that level of tensioning by closing the locking member 208 on the rotating wheel 204. The locking member 208 may optionally lock the rotating wheel 204 at any desired angle, and thereby at any desired level of tension on the tensioning suture 220.

The above-described drainage catheters with locking hubs allow the user to securely lock the pigtail of the tubular body with only one hand or even directly hold and pull the tensioning suture. Known drainage catheter locking hubs may only run distally through the lumen to the pigtail region where the end of the suture is anchored. The present drainage catheter with locking hub can terminate both ends of the tensioning suture at the locking hub, which can facilitate one-handed locking in that it cuts in half the amount of travel required at the hub to fully tighten and secure the suture at the pigtail end. Because the suture is attached to the actuation member, the suture does not need to be touched or trimmed at the completion of the procedure as with current devices. The present drainage catheter can be unlocked, repositioned and re-locked at a later date without having to remove the catheter from the drainage location and/or provide a new drainage catheter.

Embodiments can be described with reference to the following numbered clauses, with certain features laid out in the dependent clauses:

Clause 1: A drainage catheter comprising: an elongated tubular body comprising a flexible distal portion and a proximal portion; a locking hub assembly comprising: a connecting member; and an actuation member movably connected to the connecting member such that one or both of the actuation member and the connecting member move relative to the other of the actuation member and the connecting member; and a tensioning suture that extends through a drainage lumen of the tubular body and connected to the flexible distal portion; wherein opposite ends of the tensioning suture are connected to the actuation member such that movement of the one or both of the actuation member and the connecting member toward the other of the actuation member and the connecting member from an unlocked configuration to a locked configuration draws the opposite ends proximally relative to the flexible distal portion thereby causing the flexible distal end to move from a loosely curled configuration to a tightly curled configuration.

Clause 2: The drainage catheter of clause 1, wherein the actuation member comprises an outer sliding shell and the connecting member is slidably received within an actuation channel of the outer sliding shell, wherein the actuation member slides relative to the connecting member in a telescoping fashion.

Clause 3: The drainage catheter of clause 2, wherein the connecting member comprises an inner sliding shell slidingly disposed in the actuation channel of the outer sliding shell, the inner sliding shell having a suture hole in communication with an inner drainage channel of the inner sliding shell.

Clause 4: The drainage catheter of clause 3, wherein the tensioning suture extends from the distal portion of the tube body, through the inner drainage channel and is threaded through the suture hole providing an anchor location for the tensioning suture.

Clause 5: The drainage catheter of clause 4, wherein the suture hole is a first suture hole, the anchor location being a first anchoring location, and one of the opposite ends being threaded through the first suture hole, the inner sliding shell having a second suture hole in communication with the inner drainage channel of the inner sliding shell, the other of the opposite ends of the tensioning suture threaded through the second suture hole providing a second anchor location for the tensioning suture.

Clause 6: The drainage catheter of clause 5, wherein movement of the actuation member toward the connecting member increases a length of the tensioning suture extending proximally from the first and second anchoring locations.

Clause 7: The drainage catheter of clause 4 or 5, wherein the actuation member comprises a strain relief member connected to a distal end of the outer sliding shell that receives the proximal portion of the tube body, wherein the strain relief member seals the suture hole in the locked configuration.

Clause 8: The drainage catheter of clause 1, wherein the connecting member comprises a shell that comprises a sliding track that extends longitudinally between a proximal end of the shell and a distal end of the shell, the actuation member comprising a sliding ring that is slidably connected to the sliding track such that the sliding ring is configured to move proximally away from the tubular body.

Clause 9: The drainage catheter of clause 8, wherein the shell has a suture hole that is in communication with a drainage channel of the shell, the tensioning suture threaded through the suture hole providing an anchoring location for the tensioning suture.

Clause 10: The drainage catheter of clause 9, wherein the anchoring location is a first anchoring location, the sliding track comprising an anchoring bar that extends laterally across the sliding track, the tensioning suture extending around the anchoring bar providing a second anchoring location for the tensioning suture.

Clause 11: The drainage catheter of clause 9 or 10, wherein the actuation member is configured to seal the suture hole in the locked configuration.

Clause 12: The drainage catheter of clause 1, wherein the actuating member comprises a rotating wheel that is rotatably connected to a shell of the connecting member.

Clause 13: The drainage catheter of clause 12, wherein the shell having a suture hole that is in communication with a drainage channel of the shell, the tensioning suture threaded through the suture hole providing an anchoring location for the tensioning suture.

Clause 14: The drainage catheter of any of clauses 1-13, wherein the opposite ends of the tensioning suture are embedded within an outer shell of the actuation member.

Clause 15: A drainage catheter comprising: an elongated tubular body comprising a flexible distal portion and a proximal portion; a locking hub assembly comprising: a connecting member; and an actuation member movably connected to the connecting member such that one or both of the actuation member and the connecting member move relative to the other of the actuation member and the connecting member; and a tensioning suture that extends through a drainage lumen of the tubular body and connected to the flexible distal portion; wherein the connecting member comprising a shell having a suture hole extending therethrough that is in communication with a drainage channel of the shell, the tensioning member threaded through the suture hole providing an anchoring location for the tensioning suture; wherein an end of the tensioning suture is connected to the actuation member such that movement of the one or both of the actuation member and the connecting member toward the other of the actuation member and the connecting member from an unlocked configuration to a locked configuration draws the end proximally relative to the flexible distal portion thereby causing the flexible distal end to move from a loosely curled configuration to a tightly curled configuration.

Clause 16: The drainage catheter of clause 15, wherein the actuation member comprises an outer sliding shell and the connecting member is slidably received within an actuation channel of the outer sliding shell, wherein the actuation member slides relative to the connecting member in a telescoping fashion.

Clause 17: The drainage catheter of clause 16, wherein movement of the actuation member toward the connecting member increases a length of the tensioning suture extending proximally from the anchoring location.

Clause 18: The drainage catheter of clause 16 or 17, wherein the actuation member comprises a strain relief member connected to a distal end of the outer sliding shell that receives the proximal portion of the tube body, wherein the strain relief member seals the suture hole in the locked configuration.

Clause 19: The drainage catheter of clause 15, wherein the connecting member comprises a shell that comprises a sliding track that extends longitudinally between a proximal end of the shell and a distal end of the shell, the actuation member comprising a sliding ring that is slidably connected to the sliding track such that the sliding ring is configured to move proximally away from the tubular body.

Clause 20: The drainage catheter of clause 15, wherein the actuating member comprises a rotating wheel that is rotatably connected to the shell of the connecting member.

It will be apparent to those skilled in the art that various modifications and variations can be made to the embodiments described herein without departing from the spirit and scope of the claimed subject matter. Thus it is intended that the specification cover the modifications and variations of the various embodiments described herein provided such modification and variations come within the scope of the appended claims and their equivalents.

What is claimed is:

1. A drainage catheter comprising:
an elongated tubular body comprising a flexible distal portion and a proximal portion;
a locking hub assembly comprising:
a connecting member; and
an actuation member movably connected to the connecting member such that one or both of the actuation member and the connecting member move relative to the other of the actuation member and the connecting member; and
a tensioning suture that extends through a drainage lumen of the tubular body and connected to the flexible distal portion;
wherein opposite ends of the tensioning suture are connected to the actuation member such that movement of the one or both of the actuation member and the connecting member toward the other of the actuation member and the connecting member from an unlocked configuration to a locked configuration draws the opposite ends proximally relative to the flexible distal portion thereby causing the flexible distal end to move from a first configuration to a second configuration.

2. The drainage catheter of claim 1, wherein the actuation member comprises an outer sliding shell and the connecting member is slidably received within an actuation channel of the outer sliding shell, wherein the actuation member slides relative to the connecting member in a telescoping fashion.

3. The drainage catheter of claim 2, wherein the connecting member comprises an inner sliding shell slidingly disposed in the actuation channel of the outer sliding shell, the inner sliding shell having a suture hole in communication with an inner drainage channel of the inner sliding shell.

4. The drainage catheter of claim 3, wherein the tensioning suture extends from the distal portion of the tube body, through the inner drainage channel and is threaded through the suture hole providing an anchor location for the tensioning suture.

5. The drainage catheter of claim 4, wherein the suture hole is a first suture hole, the anchor location being a first anchoring location, and one of the opposite ends being threaded through the first suture hole, the inner sliding shell having a second suture hole in communication with the inner drainage channel of the inner sliding shell, the other of the opposite ends of the tensioning suture threaded through the second suture hole providing a second anchor location for the tensioning suture.

6. The drainage catheter of claim 5, wherein movement of the actuation member toward the connecting member increases a length of the tensioning suture extending proximally from the first and second anchoring locations, wherein the first configuration of the flexible distal end is a loosely curled configuration and the second configuration of the flexible distal end is a tightly curled configuration.

7. The drainage catheter of claim 4, wherein the actuation member comprises a strain relief member connected to a distal end of the outer sliding shell that receives the proximal portion of the tube body, wherein the strain relief member seals the suture hole in the locked configuration.

8. The drainage catheter of claim 1, wherein the connecting member comprises a shell that comprises a sliding track that extends longitudinally between a proximal end of the shell and a distal end of the shell, the actuation member comprising a sliding ring that is slidably connected to the sliding track such that the sliding ring is configured to move proximally away from the tubular body.

9. The drainage catheter of claim 8, wherein the shell has a suture hole that is in communication with a drainage channel of the shell, the tensioning suture threaded through the suture hole providing an anchoring location for the tensioning suture.

10. The drainage catheter of claim 9, wherein the anchoring location is a first anchoring location, the sliding track comprising an anchoring bar that extends laterally across the sliding track, the tensioning suture extending around the anchoring bar providing a second anchoring location for the tensioning suture.

11. The drainage catheter of claim 9, wherein the actuation member is configured to seal the suture hole in the locked configuration.

12. The drainage catheter of claim 1, wherein the actuating member comprises a rotating wheel that is rotatably connected to a shell of the connecting member.

13. The drainage catheter of claim 12, wherein the shell having a suture hole that is in communication with a drainage channel of the shell, the tensioning suture threaded through the suture hole providing an anchoring location for the tensioning suture.

14. The drainage catheter of claim 1, wherein the opposite ends of the tensioning suture are embedded within an outer shell of the actuation member.

15. A drainage catheter comprising:
an elongated tubular body comprising a flexible distal portion and a proximal portion;
a locking hub assembly comprising:
a connecting member; and
an actuation member movably connected to the connecting member such that one or both of the actuation member and the connecting member move relative to the other of the actuation member and the connecting member; and
a tensioning suture that extends through a drainage lumen of the tubular body and connected to the flexible distal portion;
wherein the connecting member comprising a shell having a suture hole extending therethrough that is in communication with a drainage channel of the shell, the tensioning member threaded through the suture hole providing an anchoring location for the tensioning suture;
wherein an end of the tensioning suture is connected to the actuation member such that movement of the one or both of the actuation member and the connecting member toward the other of the actuation member and the connecting member from an unlocked configuration to a locked configuration draws the end proximally relative to the flexible distal portion thereby causing the flexible distal end to move from a first configuration to a second configuration.

16. The drainage catheter of claim 15, wherein the actuation member comprises an outer sliding shell and the connecting member is slidably received within an actuation channel of the outer sliding shell, wherein the actuation member slides relative to the connecting member in a telescoping fashion.

17. The drainage catheter of claim 16, wherein movement of the actuation member toward the connecting member increases a length of the tensioning suture extending proximally from the anchoring location.

18. The drainage catheter of claim 16, wherein the actuation member comprises a strain relief member connected to a distal end of the outer sliding shell that receives the proximal portion of the tube body, wherein the strain relief member seals the suture hole in the locked configuration.

19. The drainage catheter of claim 15, wherein the connecting member comprises a shell that comprises a sliding track that extends longitudinally between a proximal end of the shell and a distal end of the shell, the actuation member comprising a sliding ring that is slidably connected to the sliding track such that the sliding ring is configured to move proximally away from the tubular body.

20. The drainage catheter of claim 15, wherein the actuating member comprises a rotating wheel that is rotatably connected to the shell of the connecting member.
